(19) United States
(12) Patent Application Publication
(10) Pub. No.: US 2008/0260871 A1
(43) Pub. Date: Oct. 23, 2008

(54) METHOD AND COMPOSITION FOR INFUSING HERBAL EXTRACTS AND PHARMACEUTICALS THROUGH THE SKIN

(76) Inventor: Clinton O. Fruitman, Chandler, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/736,905

(22) Filed: Apr. 18, 2007

(51) Int. Cl. A61K 36/886; A61K 31/10; A61K 47/00; A61P 23/00; A61P 25/04

(52) U.S. Cl. 424/744; 514/711; 514/785

(57) ABSTRACT

A topical mixture, that when massaged onto the skin, enables infusion of drugs or herbal compounds into deeper tissues, and a method of enhancing the penetration and concentration of drugs and herbs into tissues, especially as relates to treatment of pain.

### FIELD OF INVENTION

This invention relates, generally, to a method and a preparation for infusing herbal and pharmaceutical devices through the skin for physiological treatments of disorders such as joint and muscle pain, inflammation from fractures, skin disorders such as psoriasis, and for cosmetic needs such as wrinkle reducers and moisturizers. More particularly, the present invention relates to the application of creams and salves that contain compounds which when massaged into the skin, will solubilize the lipidenous and waxy deposits in the normally waterproof SGR (Stratum Granulosum) layer of the skin such that aqueous and non aqueous dissolved herbal and pharmaceutical compounds can pass the SGR barrier and be taken up and diffuse with serum beneath the skin.

### BACKGROUND OF THE INVENTION

Many herbal and pharmaceutical topical remedies exist in the marketplace for conditions such as pain, inflammation and swelling. These remedies would work well if they could be effectively absorbed and transported into deep tissues or joints where acute injuries or chronic conditions persist. Unfortunately, most topical remedies offer very limited ability to penetrate the SGR layer of the skin which functions as a waterproof barrier. Examples of this include herbal creams containing Aloe Vera and Methylsulfonylmethane (MSM) compounds. While Aloe Vera and MSM compounds are widely recognized for their anti-inflammatory, analgesic, and healing properties, these existing topical preparations have limited absorption capabilities. As a result, the producers of these preparations add agents such as menthol and pepper extracts such as capsaicin (see, for example, U.S. Pat. No. 5,665,378) which provide pain relief primarily by creating heat which produces a warming sensation at the treated site.

Compositions and compounds which do not necessarily limit absorption into the skin include (i) triethanolamines in creams such as those used with aspirin cream compounds, (ii) polyvinylpyrrolidone, a polymer which enhances wetting of the Stratum Corneum (SC) and perhaps the subcutaneous layer of the skin (see, for example, U.S. Pat. No. 5,614,212), (iii) phospholipids and at least one polyoxyethylenepolyoxypropylene copolymer (see, for example, U.S. Pat. No. 5,885,597), (iv) dimethyl sulfoxide (DMSO) preparations, (v) ethoxylated oils used in some wrinkle creams and pharmaceuticals (see, for example, U.S. Pat. Nos., 4,749,517, 5,318,960, 6,103,770 and 6,946,144), (vi) Emu oil which has some acceptance as a transdermal carrier based on its ability to wet skin and soften lipids (see, for example, U.S. Pat. Nos. 5,431,924, 5,958,384, 5,849,334, and 6,103,246), and (vii) Aloe Vera which is also considered by some to have transdermal carrying capacity for hormonal preparations (see, for example, U.S. Pat. No. 5,708,038). Since Aloe Vera contains many hydrophilic compounds, such as enzymes, amino acids and carbohydrates, as well as hydrophobic compounds, such as vitamins and sterols, some studies postulate that pharmacologic agents of both solubilities can be placed in Aloe Vera and carried through the epidermal barrier.

Because DMSO and Ethoxylated oils (particularly Ethoxylated Esters) offer limited solubilities of both lipids and aqueous phase compounds, they can be useful as transdermal carriers that penetrate the Stratum Corneum (SC) and SGR layers of the skin. Other carriers such as phospholipids and polymers are useful in helping transport non aqueous compounds through the SC layer of the skin.

However, drawbacks exist with all of the prior art compositions. These drawbacks include poor efficacy of many conventional oil/water creams and salves, the solubility limitations of ethoxylated oils, and the legal ramifications in the United States of preparations utilizing DMSO. Accordingly, there is a need for a method and composition for infusing herbs and pharmaceuticals through the skin which enables the composition to pass the SGR layer of the skin and deliver the herbs and pharmaceuticals beneath the skin so they can be taken up by serum and delivered to deep tissue beneath the skin.

### SUMMARY OF THE INVENTION

In general, the present invention provides a method of infusing herbal compounds and drugs through the skin and into subcutaneous tissue by rubbing the skin with a preparation that includes natural herbal oils that have the potential to dissolve and soften the lipids and waxy deposits in the skin. The preparation typically comprises an aqueous mixture of herbs or drugs.

In one exemplary embodiment, the composition comprises at least one of a solubilizing oil and a solubilizing grease which comprises about 30% by weight of the composition, and an aqueous phase having at least one of an herb and pharmaceutical which comprises about 70% by weight of the composition. The aqueous phase may include refined Aloe Vera juice and 10% by weight dissolved methylsulfonylmethane.

In a further exemplary embodiment the solubilizing oil is comprised of Jojoba esters which have a remarkable ability of penetrating, as well as dissolving through the SGR layer of the skin due to the slippery nature of their long aliphatic carbon chains, 16 to 22 carbons on each side of the ester linkage.

The preparation, typically a cream, is massaged into the skin until the skin is almost dry. Some oily residue may remain, but the aqueous phase is largely absorbed both through the softening and dissolution of the SGR lipids combined with the hydraulic pressure of rubbing on the aqueous micro-droplets in the cream. The act of massaging the cream into the skin has the added benefit of concentrating the absorption into tissues and joints where needed. In addition, massage is also therapeutic to affected areas.

In another exemplary embodiment, a cold pack is held over the area of the skin where the composition has penetrated as a result of rubbing the composition into the skin.

### BRIEF DESCRIPTION OF THE DRAWINGS

FIG. 1 is an illustration of the mode of action of the method and composition of the present invention for infusing herbs and pharmaceuticals through the skin.

### DETAILED DESCRIPTION OF THE EXEMPLARY EMBODIMENTS

The present invention is directed to a composition for infusing herbs and pharmaceuticals through the skin which includes a solubilizing oil or grease and an aqueous phase that includes an herb and/or a pharmaceutical.

In one preferred embodiment, the cream consists of about 30 wt. % Simmondsia (Jojoba) Oil and a 70 w. % aqueous phase where the aqueous phase consists of refined Aloe Vera juice and 10 wt. % dissolved MSM. The cream is stabilized with Carbopol, a typical Carbomer suspending agent, and the pH is controlled between 7.5 and 8 with Sodium Bicarbonate. The method for infusing medications and/or herbs through the skin and into subcutaneous tissue includes rubbing the composition, or cream, into the skin to penetrate the waterproof skin barrier. The cream is liberally massaged into painful areas of the body such as the neck, back, elbows, arms, hands, hips, knees, and feet.

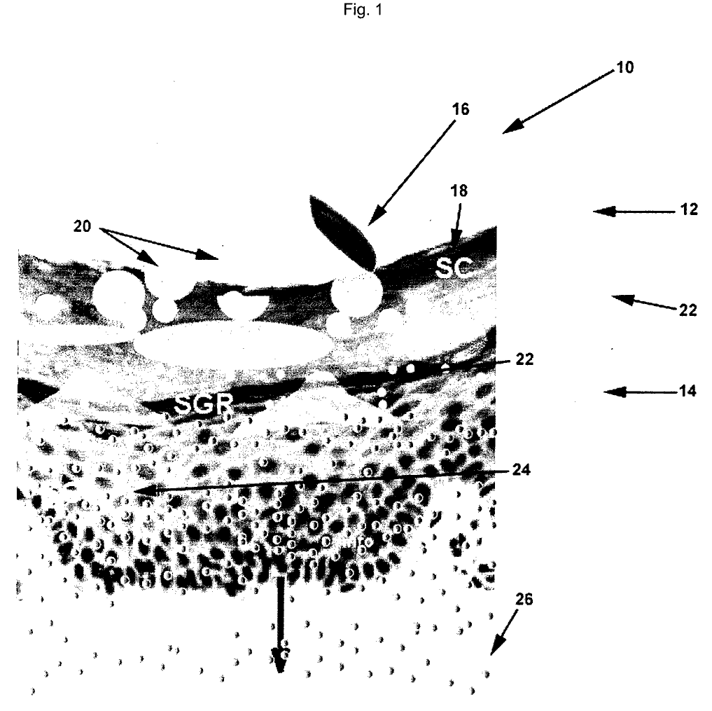

FIG. 1 illustrates the mode of action 10 of the present invention which involves rubbing a composition, such as a cream, containing at least one of a solubilizing grease or solubilizing oil and an aqueous phase having an herb and/or pharmaceutical into the skin. The composition of the present invention includes micro droplets of oil 12 in the oil phase of the cream and micro droplets of water 14 containing herbal extracts or pharmaceuticals (i.e. medications) in the aqueous phase of the cream.

The cream is rubbed onto the skin using pressure applied by a finger 16 and the aqueous and oil phases of the cream are pressure infused through the top layer of the skin's porous dead cells or Stratum Corneum (SC) 18 by massage. The oil phase and aqueous phase in the cream are forced into the skin as shown at reference number 20. The lipid components of the SGR layer of the skin are dissolved and the SGR layer is softened from the wetting action of the oil phase as shown by reference numeral 22.

The Stratum Germinativurn (SG) layer 24 is where live skin cells evolve and are nourished by diffusing serum. Here the serum takes up the infused herbal extracts or pharmaceuticals and transports them to deeper tissues. Meanwhile, the hydraulic pressure enhanced infusion moves the aqueous phase through the skin into serum of subcutaneous areas and deeper into the tissues as shown in reference numeral 26.

In another exemplary embodiment of the present invention, after the Oil/Aqueous cream containing herbs or drugs is massaged into the skin, the affected area is packed with a cold pad for fifteen minutes. The cold tends to cause dis-inflammation and shrinkage of affected tissues. In addition to the physical medicine benefits of cold packing, there are two other benefits, namely 1) The distance that subcutaneous drugs or herbs must diffuse to reach affected areas is reduced with the reduction in swelling and 2) as cold the temperature causes serum in tissues beneath the skin to draw away from the contact area, the dissolved drugs or herbs are drawn deeper into the tissues and joints and concentrated in the serum at the needed sites.

Methods and results used to evaluate the method and composition of the present invention for infusing herbs and pharmaceuticals through the skin are set out below.

### Methods

A hundred volunteers were asked to score their pain on a scale of 1 to 10 before and after self treatment (with the exemplary embodiments described above) at the end of 15 minutes, 4 hr. and 8 hr.

In some cases infrared temperature measurements were made on the skin over painful areas before and immediately after treatments. Places of injury or chronic pain frequently radiate higher levels of heat than normal tissue.

The volunteers presented themselves with multiple locations of chronic and acute pain ranging in severity from 3 to 9. These included severe painful deformations of the feet such as Charcot Foot, knee deterioration, Carpal type hand pain and immobility, back pain, neck pain, shoulder pain, muscle spasms, un-cast stress fractured ulna, stress fractured metatarsals, and hip pain. The infrared measured skin temperatures over painful areas were typically 4 to 5 degrees F. higher than adjacent normal tissue.

### Treatment Results

After a five minute massage with a cream as described in the first preferred embodiment, 96% of the volunteers felt relief ranging from 60% to 90%. The skin temperature over the affected areas dropped an average of 4 degrees.

Following the cream massage, the painful areas were packed for fifteen minutes with—5 degree F. cold soft rubber pads placed directly on the skin as described in published Patent application no. 20060142816. Ninety-six percent of the volunteers felt relief ranging from 80% to 100%. Skin temperatures over the affected areas averaged 60 degrees F.

In the case of the fractured Ulna—initially recorded as a pain severity of 8, the volunteer felt no relief and repeated both treatments. At the end of the second treatment, the severity was recorded as 1.

In one case of level 6 chronic knee pain, the volunteer felt no initial relief, but reported back the next day that after 8 hours the pain diminished and he was still pain free 16 hours later.

Of the 97% of volunteers who reported initial pain relief of 80 to 100%, the relief lasted between four and eight hours with half of the volunteers reporting lower pain scores at eight hours.

In further evaluations, through the physical therapy department of a large nursing home, the chronic pain and acute post surgical pain patients with back, neck, foot, hand, or limb complaints have been managed with the cream massage followed by cold packing. These patients typically have pain between 6 and 8. After treatment their pain is typically 1 to 2 and with very few exceptions, this treatment has enabled patients to decrease their use of narcotic medications significantly.

The described embodiments are to be considered in all respects only as illustrative of the current best modes of the invention known to the inventor at the time of filing this application, and not as restrictive. Although the several embodiments shown here include specific components, these are provided in order to show examples of the present embodiments of this invention. The specifics of these embodiments are provided to show several examples. This scope of this invention is, therefore, indicated by the appended claims rather than by the foregoing description. All devices and processes that come within the meaning and range of equivalency of the claims are to be embraced as with the scope of this patent.

1. A composition for infusing herbs and pharmaceuticals through the skin for physiological treatments comprising:
at least one of a solubilizing oil and a solubilizing grease; and
an aqueous phase which includes at least one of an herb and a pharmaceutical.

2. The composition of claim 1 wherein the solubilizing oil is comprised of an ester.

3. The composition of claim 1 wherein the solubilizing oil is dispersed within the aqueous phase.

4. The composition of claim 1 wherein the composition comprises a cream or a salve.

5. The composition of claim 1 wherein the pharmaceutical comprises at least one of an analgesic and an anesthetic.

6. The composition of claim 1 wherein the aqueous phase comprises Aloe Vera extracts.

7. The composition of claim 1 wherein the aqueous phase comprises methylsulfonylmethane.

8. The composition of claim 1 wherein said at least one of a solubilizing oil and a solubilizing grease comprises about 30% weight of the composition and said aqueous phase comprises about 70% weight of the composition.

9. The composition of claim 7 wherein the aqueous phase comprises refined Aloe Vera juice and 10% weight dissolved methylsulfonylmethane.

10. A method for infusing medications or herbal compounds through the skin and into subcutaneous tissues comprising:
providing a composition comprising at least one of a solubilizing oil and a solubilizing grease and an aqueous phase which includes at least one of an herb and a pharmaceutical; and
rubbing the composition into the skin to penetrate the waterproof skin barrier and diffuse into deeper tissue with the underlying serum.

11. The method of claim 10 further comprising the step of positioning a cold pack over the area of skin where the composition has been penetrated.

12. The method of claim 10 wherein the step of providing a composition comprises the step of providing a composition wherein the solubilizing oil is dispersed within the aqueous phase.

13. The method of claim 10 wherein the step of providing a composition comprises the step of providing a composition wherein the composition comprises a cream or a salve.

14. The method of claim 10 wherein the step of providing a composition comprises the step of providing a composition wherein the pharmaceutical comprises at least one of an analgesic and an anesthetic.

15. The method of claim 10 wherein the step of providing a composition comprises the step of providing a composition wherein the aqueous phase comprises Aloe Vera extracts.

16. The method of claim 10 wherein the step of providing a composition comprises the step of providing a composition wherein the aqueous phase comprises methylsulfonylmethane.

17. The method of claim 10 wherein the step of providing a composition comprises the step of providing a composition wherein said at least one of a solubilizing oil and a solubilizing grease comprises about 30% weight of the composition and said aqueous phase comprises about 70% weight of the composition.

18. The method of claim 17 wherein the step of providing a composition comprises the step of providing a composition wherein the aqueous phase comprises Aloe Vera juice and 10% weight dissolved methylsulfonylmethane.
